(19) United States
(12) Patent Application Publication
(10) Pub. No.: US 2018/0049630 A1
(43) Pub. Date: Feb. 22, 2018

(54) ENDOSCOPE SYSTEM

(71) Applicant: HOYA CORPORATION, Tokyo (JP)

(72) Inventors: Hidenori TAKUSHIMA, Saitama (JP); Kunihiko ONOBORI, Tokyo (JP); Fumika YOKOUCHI, Tokyo (JP)

(73) Assignee: HOYA CORPORATION, Tokyo (JP)

(21) Appl. No.: 15/552,605

(22) PCT Filed: Nov. 15, 2016
(86) PCT No.: PCT/JP2016/083768

§ 371 Date: Aug. 22, 2017

(30) Foreign Application Priority Data
Dec. 10, 2015 (JP) 2015-241621

(51) Int. Cl. A61B 1/06; A61B 1/00; F21V 14/08; F21V 23/00; F21V 9/08; A61B 1/07; A61B 1/04

(52) U.S. Cl. CPC A61B 1/0638; A61B 1/0684; A61B 1/00004; F21V 14/08; H04N 2005/2255; F21V 9/08; A61B 1/07; A61B 1/04; A61B 1/00045; F21V 23/003

(57) ABSTRACT

An endoscope system includes: a first light source that emits first light; a first light guide that guides the first light received from the first light source toward a subject; a second light source that emits second light having a different wavelength region from the first light; a second light guide that guides the second light received from the second light source toward the subject; and a blocker that alternatingly blocks the first light traveling from the first light source toward the first light guide and the second light traveling from the second light source toward the second light guide.

### TECHNICAL FIELD

The present invention relates to an endoscope system that irradiates a subject with light.

### BACKGROUND ART

Endoscope systems that can capture special images are known. A specific configuration of this type of endoscope system is disclosed in WO 2012/108420 pamphlet (called “Patent Document 1” hereinafter), for example.

The endoscope system disclosed in Patent Document 1 includes a light source apparatus that is provided with a rotating filter. The rotating filter is an optical filter that allows only light in a specific wavelength region to pass, and rather than having a simple disk shape; has a special shape in which a portion of the outer circumferential region is cut away. A controller drives the rotating filter to rotate at a constant rotation period such that the optical filter portion and the cutaway portion successively enter the light path of irradiation light, and an image of biological tissue formed by irradiation light that passed through the optical filter portion and an image of biological tissue formed by irradiation light that passed through the cutaway portion (i.e., unfiltered irradiation light) are successively captured. The controller generates one observation image based on captured image data regarding the biological tissue irradiated by irradiation light that passed through the optical filter portion, generates another observation image based on captured image data regarding biological tissue illuminated with unfiltered irradiation light, and displays these two types of generated observation images side-by-side on the display screen of a monitor.

### SUMMARY OF INVENTION

Silk lines for detecting the rotation position of the rotating filter are printed on the central portion of the rotating filter disclosed in Patent Document 1. However, the silk lines are extremely small, and therefore there is a problem in that the rotation position of the rotating filter cannot be precisely detected if there is even a slight error in the silk lines.

The present invention was achieved in light of the above-described circumstances, and an object of the present invention is to provide an endoscope system that is suited to irradiating a subject with two types of irradiation light that have different wavelength regions.

An endoscope system according to one embodiment of the present invention includes: a first light source portion that emits first light; a first light guiding member that guides the first light received from the first light source portion toward a subject; a second light source portion that emits second light having a different wavelength region from the first light; a second light guiding member that guides the second light received from the second light source portion toward the subject; and a blocking portion that alternatingly blocks the first light traveling from the first light source portion toward the first light guiding member and the second light traveling from the second light source portion toward the second light guiding member.

Also, in the endoscope system according to the embodiment of the present invention, a configuration is possible in which the blocking portion alternatingly blocks the first light and the second light in accordance with a timing synchronized with a predetermined imaging cycle.

Also, an endoscope system according to one embodiment of the present invention includes: a first light source portion that emits first light; a first light guiding member that guides the first light received from the first light source portion toward a subject; a second light source portion that emits second light having a different wavelength region from the first light; a second light guiding member that guides the second light received from the second light source portion toward the subject; and a control portion that, by alternatingly turning on a light source of the first light source portion and a light source of the second light source portion, alternatingly allows the first light and the second light to enter the first light guiding member and the second light guiding member.

Also, in the endoscope system according to the embodiment of the present invention, a configuration is possible in which the control portion alternatingly turns on and off the light source of the first light source portion and the light source of the second light source portion in accordance with a timing synchronized with a predetermined imaging cycle.

Also, in the endoscope system according to the embodiment of the present invention, a configuration is possible in which the first light source portion has a light source that emits the first light, and the second light source portion has a light source that emits third light, and an optical filter that filters the third light to obtain the second light.

According to the embodiment of the present invention, an endoscope system that is suited to irradiating a subject with two types of irradiation light that have different wavelength regions is provided.

### BRIEF DESCRIPTION OF DRAWINGS

FIG. 1 is a block diagram showing a configuration of an electronic endoscope system according to an embodiment of the present invention.

FIG. 2 is a diagram showing a spectral intensity distribution of LEDs included in the electronic endoscope system of the embodiment of the present invention.

FIG. 3 is a diagram showing spectral characteristics of a narrow-band light filter included in the electronic endoscope system of the embodiment of the present invention.

FIG. 4 is a diagram showing a configuration of a shutter portion included in the electronic endoscope system of the embodiment of the present invention.

FIG. 5 is a diagram schematically showing a configuration of a connection portion of an electronic endoscope and a processor according to the embodiment of the present invention.

FIG. 6 is a block diagram showing a configuration of an electronic endoscope system according to a first variation of the embodiment of the present invention.

FIG. 7 is a block diagram showing a configuration of an electronic endoscope system according to a second variation of the embodiment of the present invention.

FIG. 8 is a block diagram showing a configuration of an electronic endoscope system according to a third variation of the embodiment of the present invention.

FIG. 9 is a block diagram showing a configuration of an electronic endoscope system according to a fourth variation of the embodiment of the present invention.

### DESCRIPTION OF EMBODIMENTS

Hereinafter, an embodiment of the present invention will be described with reference to the drawings. Note that an electronic endoscope system is taken as an example of an embodiment of the present invention in the following description.

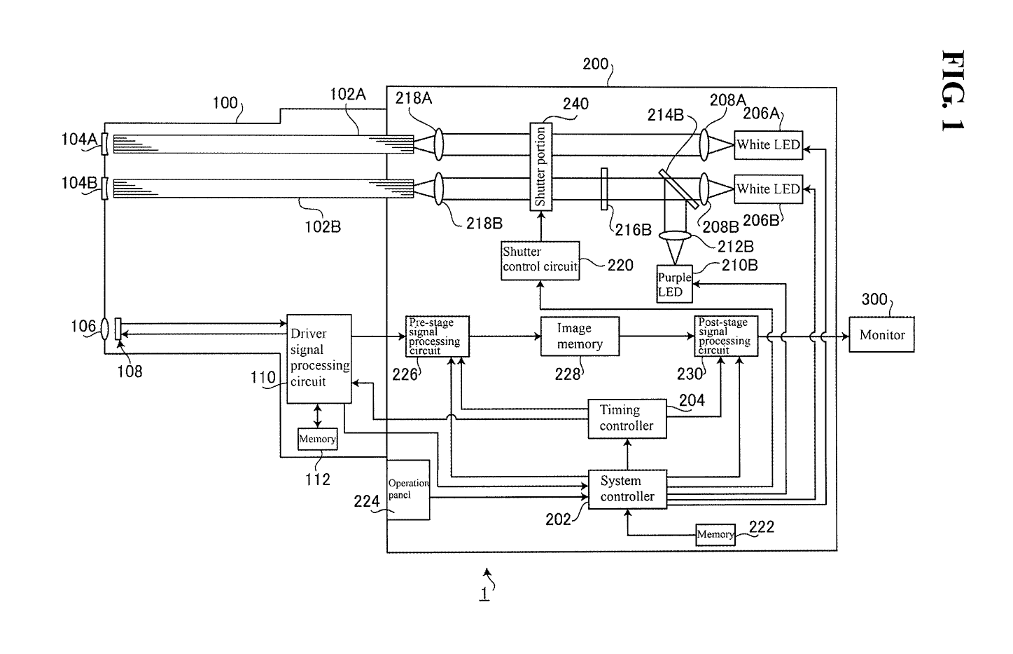

FIG. 1 is a block diagram showing the configuration of an electronic endoscope system 1 according to an embodiment of the present invention. As shown in FIG. 1, the electronic endoscope system 1 is a system specialized for medical use, and includes an electronic endoscope 100, a processor 200, and a monitor 300.

The processor 200 includes a system controller 202 and a timing controller 204. The system controller 202 executes various programs stored in a memory 222 and performs overall control of the electronic endoscope system 1. Also, the system controller 202 is connected to an operation panel 224. The system controller 202 changes operations of the electronic endoscope system 1 and parameters for various operations in accordance with instructions from an operator that are input using the operation panel 224. One example of an instruction input by an operator is an instruction for switching the observation mode of the electronic endoscope system 1. Examples of observation modes include a normal observation mode, a special observation mode, and a twin observation mode. The timing controller 204 outputs a clock pulse, which is for adjustment of the timing of the operations of portions, to circuits in the electronic endoscope system 1.

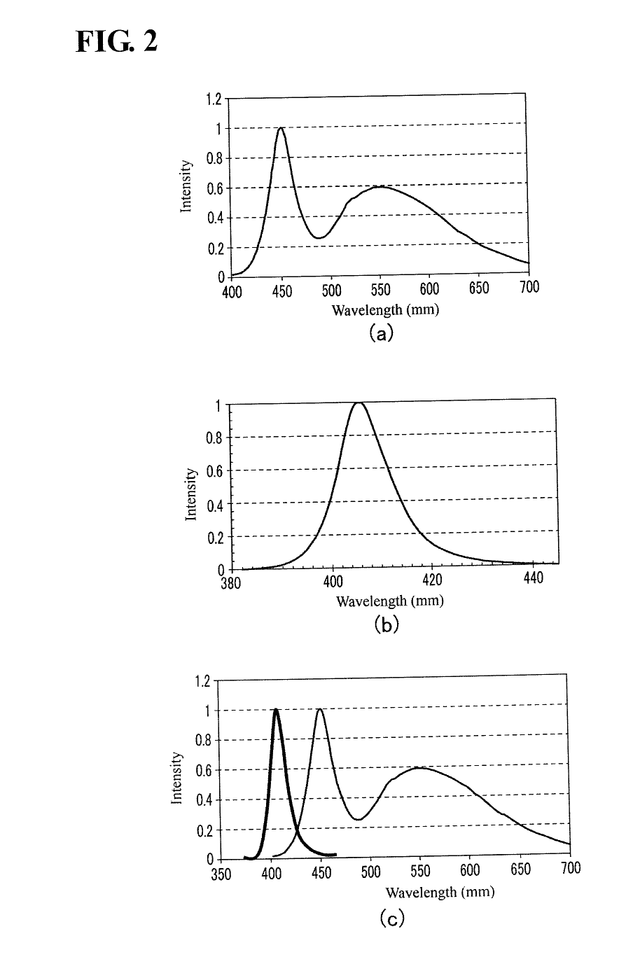

The processor 200 includes white LEDs (Light Emitting Diodes) 206A and 206B. FIG. 2(a) shows an example of the spectral intensity distribution of the white LEDs 206A and 206B. As shown in FIG. 2(a), the white LEDs 206A and 206B are so-called pseudo white light sources that have an uneven emission spectrum.

The processor 200 also includes a purple LED 210B. FIG. 2(b) shows an example of the spectral intensity distribution of the purple LED 210B. As shown in FIG. 2(b), the purple LED 210B is a light source that emits only light in the purple region.

The white LED 206A is one example of a first light source portion. White light emitted by the white LED 206A passes through a collimator lens 208A and enters a shutter portion 240.

The white LED 206B, the purple LED 210B, and a dichroic mirror 214B are one example of a second light source portion. White light emitted by the white LED 206B and purple light emitted by the purple LED 210B respectively pass through collimator lenses 208B and 212B and are incident on the dichroic mirror 214B. In other words, light that is a combination of white light and purple light (light having the spectral intensity distribution illustrated in FIG. 2(c)) is incident on the dichroic mirror 214B. Hereinafter, for the sake of convenience in the description, this light that is a combination of white light and purple light will be referred to as “superimposed light”.

The superimposed light that is incident on the dichroic mirror 214B is filtered by a narrow-band light filter 216B and then enters the shutter portion 240. Here, the narrow-band light filter 216B is attached to the case of the processor 200 and has a fixed position in the case. The narrow-band light filter 216B is shaped as a simple disk, for example.

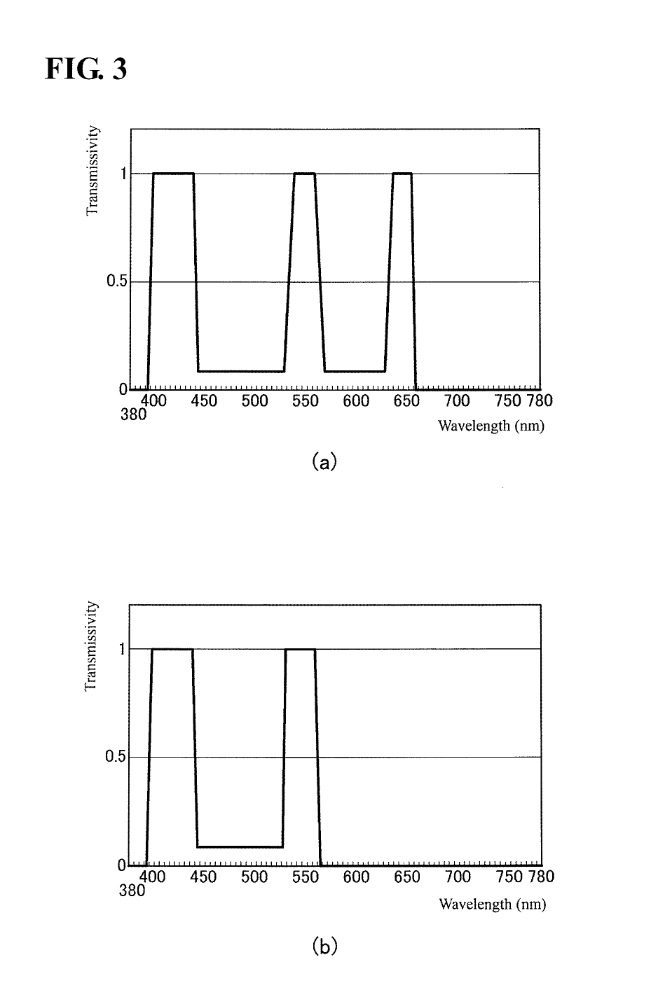

FIG. 3(a) shows an example of the spectral characteristics of the narrow-band light filter 216B. Also, FIG. 3(b) shows an example of different spectral characteristics from FIG. 3(a) for the narrow-band light filter 216B. As shown in FIGS. 3(a) and 3(b), the narrow-band light filter 216B has a spectral characteristic of allowing only light in a specific wavelength region to pass. For the sake of convenience in the description, light filtered by the narrow-band light filter 216B will be referred to as “special light”.

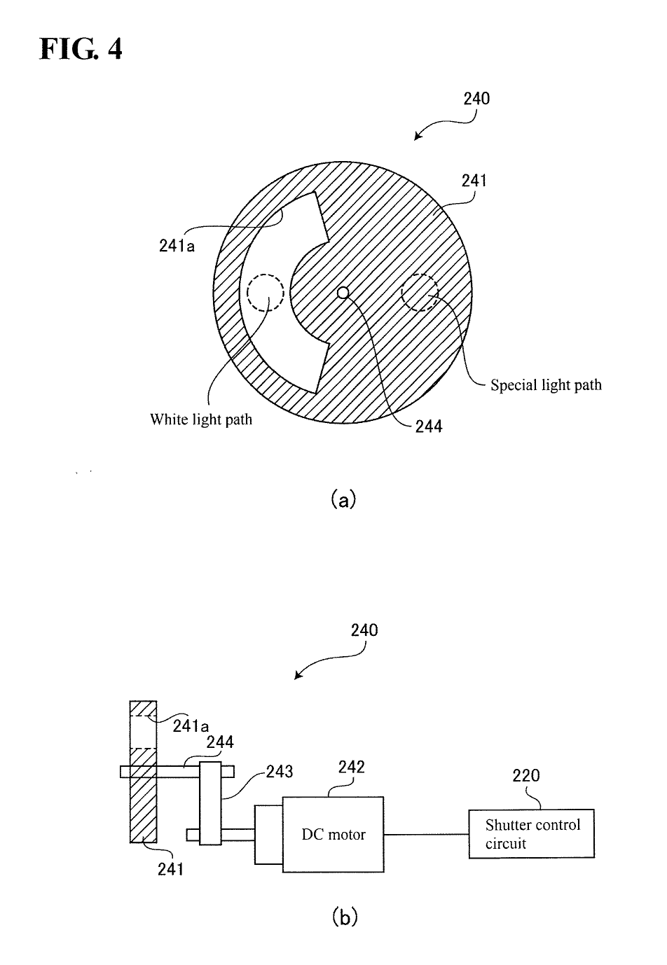

FIG. 4 shows the configuration of the shutter portion 240. The shutter portion 240 functions as a light blocking portion that alternatingly blocks light from the first light source portion and light from the second light source portion, and includes a rotating disk 241 as shown in FIG. 4. The rotating disk 241 is a member made of a metal such as stainless steel, and an opening 241a is formed therein as shown in FIG. 4(a). The opening 241a is shaped as a fan that spreads out over approximately 180°.

When a shutter control circuit 220 performs control to drive a DC motor 242 under control of the system controller 202, driving force from the DC motor 242 is transmitted to a rotation shaft 244 via a transmission mechanism (belt) 243, and thus the rotation shaft 244 rotates. Accordingly, the rotating disk 241 rotates about the rotation shaft 244.

The rotating disk 241 blocks the light path of either the white light or the special light according to its rotation position, and at the same time the opening 241a is inserted into the other light path, thereby allowing light on the other light path to pass. Hereinafter, for the sake of convenience in the description, the state where the opening 241a is located in the light path of white light (in other words, the state of blocking only the light path of special light) will be referred to as the “white light transmission state”, and the state where the opening 241a is located in the light path of special light (in other words, the state of blocking only the light path of white light) will be referred to as the “special light transmission state”.

The position of the opening 241a alternatingly switches between the light path of white light and the light path of special light due to the rotating disk 241 rotating about the rotation shaft 244. Hereinafter, for the sake of convenience in the description, during rotation of the rotating disk 241, the period in which the opening 241a is located in the light path of white light will be referred to as the “white light transmission period”, and the period in which the opening 241a is located in the light path of special light will be referred to as the “special light transmission period”. Note that the angular range of the opening 241a is slightly less than 180°, and therefore a very small blocking period in which the light paths of both white light and special light are blocked exists between the white light transmission period and the special light transmission period.

In the white light transmission period, white light that passed through the collimator lens 208A passes through the opening 241a and enters a condensing lens 218A. The white light that entered the condensing lens 218A is condensed on the entrance surface of an LCB (Light Carrying Bundle) 1.02A by the condensing lens 218A, and enters the LCB 102A.

The LCB 102A functions as a first light guiding member that guides light from the first light source portion to the subject. The white light that entered the LCB 102A propagates inside the LCB 102A. The white light that propagated inside the LCB 102A exits from the exit surface of the LCB 102A arranged at the distal end of the electronic endoscope 100, passes through a light distribution lens 104A, and irradiates the subject. Returning light from the subject irradiated by the white light from the light distribution lens 104A passes through an objective lens 106 and forms an optical image on the light receiving surface of a solid-state image sensor 108.

In the special light transmission period, special light that passed through the narrow-band light filter 216B passes through the opening 241a and enters a condensing lens 218B. The special light that entered the condensing lens 218B is condensed on the entrance surface of an LCB 102B by the condensing lens 218B, and enters the LCB 102B.

The LCB 102B functions as a second light guiding member that guides light from the second light source portion to the subject. The special light that entered the LCB 102B propagates inside the LCB 102B. The special light that propagated inside the LCB 102B exits from the exit surface of the LCB 102B arranged at the distal end of the electronic endoscope 100, passes through a light distribution lens 104B, and irradiates the subject. Returning light from the subject irradiated by the special light from the light distribution lens 104B passes through the objective lens 106 and forms an optical image on the light receiving surface of the solid-state image sensor 108.

The solid-state image sensor 108 is a single-plate color CCD (Charge Coupled Device) image sensor that has a Bayer pixel arrangement. The solid-state image sensor 108 accumulates charge according to the light quantity of an optical image formed on pixels on the light receiving surface, generates R (Red), G (Green), and B (Blue) image signals, and outputs the image signals. Note that the solid-state image sensor 108 is not limited to being a CCD image sensor, and may be replaced with a CMOS (Complementary Metal Oxide Semiconductor) image sensor or another type of imaging apparatus. The solid-state image sensor 108 may be an element that includes a complementary color filter.

A driver signal processing circuit 110 is provided in the connection portion of the electronic endoscope 100. Image signals of the subject irradiated by light from the light distribution lens 104A or the light distribution lens 104B are input by the solid-state image sensor 108 to the driver signal processing circuit 110 at a frame cycle. Note that the terms “frame” and “field” may be switched in the following description. In the present embodiment, the frame cycle and the field cycle are respectively 1/30 seconds and 1/60 seconds. The image signals input from the solid-state image sensor 108 are subjected to predetermined processing by the driver signal processing circuit 110 and output to a pre-stage signal processing circuit 226 of the processor 200.

The driver signal processing circuit 110 also accesses a memory 112 and reads out unique information regarding the electronic endoscope 100. The unique information regarding the electronic endoscope 100 recorded in the memory 112 includes, for example, the pixel count, sensitivity, operable frame rate, and model number of the solid-state image sensor 108. The unique information read out from the memory 112 is output by the driver signal processing circuit 110 to the system controller 202.

The system controller 202 generates control signals by performing various computation based on the unique information regarding the electronic endoscope 100. The system controller 202 uses the generated control signals to control the operations of and the timing of various circuits in the processor 200 so as to perform processing suited to the electronic endoscope that is connected to the processor 200.

A timing controller 204 supplies a clock pulse to the driver signal processing circuit 110 in accordance with timing control performed by the system controller 202. In accordance with the clock pulse supplied from the timing controller 204, the driver signal processing circuit 110 controls the driving of the solid-state image sensor 108 according to a timing synchronized with the frame rate of the images processed by the processor 200.

The pre-stage signal processing circuit. 226 performs predetermined signal processing such as demosaicing, processing, matrix computation, and Y/C separation on the image signal received in one frame cycle from the driver signal processing circuit 110, and outputs the result to an image memory 228.

The image memory 228 buffers image signals received from the pre-stage signal processing circuit 226, and outputs the image signals to a post-stage signal processing circuit 230 in accordance with timing control performed by the timing controller 204.

The post-stage signal processing circuit 230 performs processing on the image signals received from the image memory 228 to generate screen data for monitor display, and converts the generated monitor display screen data into a predetermined video format signal. The converted video format signal is output to the monitor 300. Accordingly, subject images are displayed on the display screen of the monitor 300.

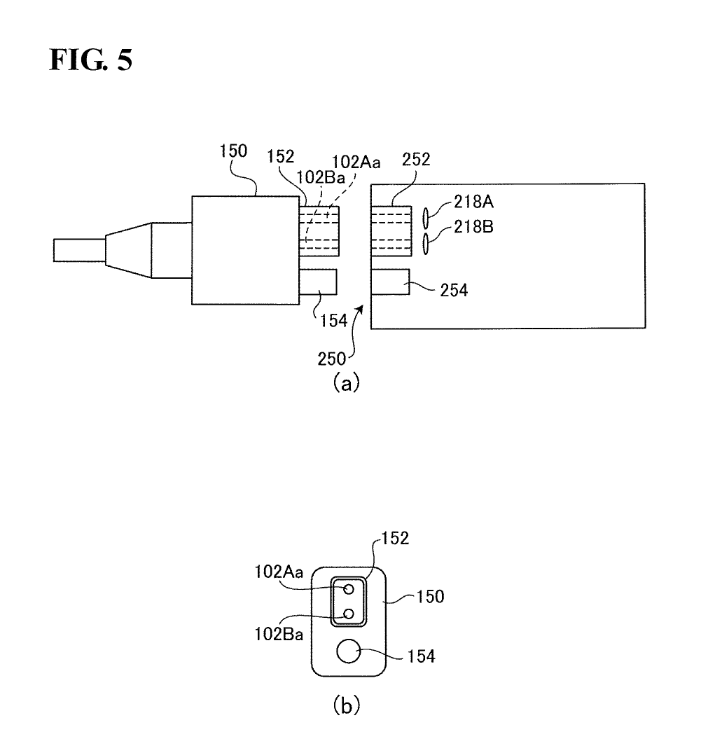

FIG. 5 is a diagram schematically showing the configuration of a connection portion of the electronic endoscope 100 and the processor 200. As shown in FIG. 5, a connector portion 150 of the electronic endoscope 100 is provided with a guide tube 152 and an electrical connector 154. The guide tube 152 holds a base end portion 102Aa of the LCB 102A and a base end portion 102Ba of the LCB 102B. Also, a connector portion 250 of the processor 200 is provided with a guide tube receiving portion 252 and an electrical connector receiving portion 254.

When the guide tube 152 and the guide tube receiving portion 252 are connected, the LCB 102A and the condensing lens 218A are coupled, and the LCB 102B and the condensing lens 218B are coupled. Accordingly, the electronic endoscope 100 and the processor 200 are optically connected. Also, when the electrical connector 154 and the electrical connector receiving portion 254 are connected, the electronic endoscope 100 and the processor 200 are electrically connected.

Next, operations of the electronic endoscope system 1 in various observation modes will be described.

Normal Observation Mode

The following describes operations of the electronic endoscope system 1 in the normal observation mode.

In the normal observation mode, the white LEDs 206A and 206B and the purple LED 210B are on at all times. Also, the rotating disk 241 is stopped in the white light transmission state. For this reason, white light emitted by the white LED 206A passes through the rotating disk 241 (opening 241a), and irradiates the subject via the condensing lens 218A, the LCB 102A, and the light distribution lens 104A. On the other hand, white light and purple light emitted by the white LED 206B and the purple LED 210B are filtered by the narrowband light filter 216B, but are blocked by the rotating disk 241 and therefore do not irradiate the subject. In other words, the subject is irradiated by white light that has the spectral intensity distribution shown in FIG. 2(a).

Note that a configuration is possible in which in the normal observation mode, the white LED 206A is on at all times, and the white LED 206B and the purple LED 210B are off at all times.

The solid-state image sensor 108 images the subject irradiated by white light, and outputs the image signal to the pre-stage signal processing circuit 226 via the driver signal processing circuit 110. The image signal is processed by the pre-stage signal processing circuit 226, the image memory 228, and the post-stage signal processing circuit 230 and then output to the monitor 300, and thus a normal color image of the subject is displayed on the display screen of the monitor 300.

Special Observation Mode

The following describes operations of the electronic endoscope system 1 in the special observation mode.

In the special observation mode, the white LEDs 206A and 206B and the purple LED 210B are on at all times. Also, the rotating disk 241 is stopped in the special light transmission state. For this reason, white light and purple light emitted by the white LED 206B and the purple LED 210B is filtered by the narrow-band light filter 216B, passes through the rotating disk 241 (opening 241a), and irradiates the subject via the condensing lens 218B, the LCB 102B, and the light distribution lens 104B. On the other hand, white light emitted by the white LED 206A is blocked by the rotating disk 241, and therefore does not irradiate the subject. In other words, the subject is irradiated by special light, which is the result of superimposed light having the spectral intensity distribution shown in FIG. 2(c) being filtered by the narrow-band light filter 216B.

Note that a configuration is possible in which in the special observation mode, the white LED 206A is off at all times, mid the white LED 206B and the purple LED 210B are on at all times.

The solid-state image sensor 108 images the subject irradiated by special light, and outputs the image signal to the pre-stage signal processing circuit 226 via the driver signal processing circuit 110. Here, this special light is light that is highly absorbed by a specific biological structure. For this reason, the image signal is processed by the pre-stage signal processing circuit 226, the image memory 228, and the post-stage signal processing circuit 230 and then output to the monitor 300, and thus a spectral image in which a specific biological structure is enhanced is displayed on the display screen of the monitor 300.

Twin Observation Mode

The following describes operations of the electronic endoscope system 1 in the twin observation mode.

In the twin observation mode, the white LEDs 206A and 206B and the purple LED 210B are on at all times. Also, the rotating disk 241 rotates about the rotation shaft 244 such that the position of the opening 241a alternatingly switches between the light path of white light and the light path of special light at a timing synchronized with the frame cycle (one frame at a time) (i.e., so as to alternatingly switch between the white light transmission period and the special light transmission period one frame at a time). For this reason, the subject is alternatingly irradiated by white light and special light at a timing synchronized with the frame cycle (one frame at a time).

In one frame, the solid-state image sensor 108 images the subject irradiated by white light and outputs the image signal to the pre-stage signal processing circuit 226 via the driver signal processing circuit 110, and then in the next frame, images the subject irradiated by special light and outputs the image signal to the pre-stage signal processing circuit 226 via the driver signal processing circuit 110. In other words, the solid-state image sensor 108 alternatingly outputs an image signal of the subject irradiated by white light and an image signal of the subject irradiated by special light to the pre-stage signal processing circuit 226 via the driver signal processing circuit 110. The former and latter image signals are processed by the pre-stage signal processing circuit 226, the image memory 228, and the post-stage signal processing circuit 230 and then output to the monitor 300.

Two regions for displaying observation images are arranged side-by-side in the display screen of the monitor 300. A normal color image of the subject irradiated by white light is displayed in one of the regions, and a spectral image in which the subject irradiated by special light (specific biological structure) is enhanced is displayed in the other region. In other words, a normal color image and a spectral image of the subject are displayed side-by-side on the display screen of the monitor 300.

In this way, according to the present embodiment, the narrow-band light filter 216B is not a moved member, but rather is a member that is fixed inside the case of the processor 200, and therefore there is no need for indicators for detecting the rotation position such as silk lines. Also, because the narrow-band light filter 216B is not a moved member, there are few constraints in terms of its shape, and it may have a simple disk shape for example. In other words, according to the present embodiment, there is no need for indicators required to have strict tolerance management, and there are few constraints on the shape of the narrow-band light filter 216B, thereby achieving advantages in terms of manufacturing (e.g., the yield is easily improved).

An illustrative embodiment of the present invention has been described above. The embodiments of the present invention are not limited to the embodiment described above, and various changes can be made without departing from the scope of the technical idea of the present invention. For example, appropriate combinations of embodiments and the like explicitly given as examples in this specification and obvious embodiments and the like are also encompassed in embodiments of the present invention.

The light source apparatus is provided inside the processor 200 in the above embodiment, but in another embodiment, a configuration is possible in which the processor 200 and the light source apparatus are separate. In this case, a wired or wireless communication means for exchanging timing signals is provided between the processor 200 and the light source apparatus.

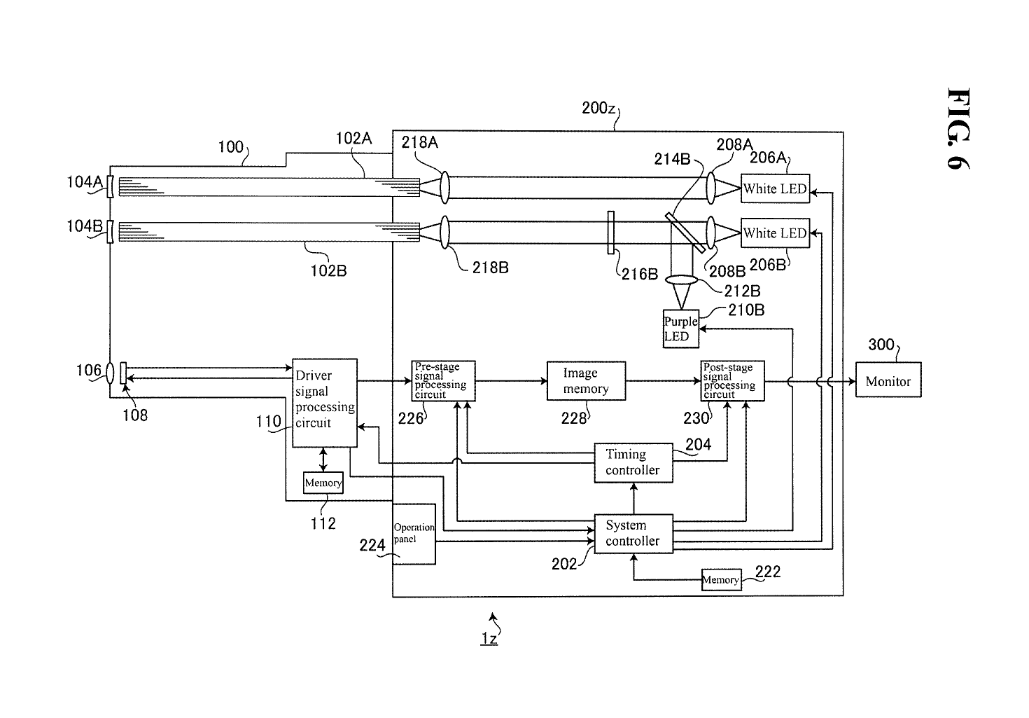

FIG. 6 is a block diagram showing the configuration of an electronic endoscope system 1z according to a first variation of the present embodiment. As shown in FIG. 6, the electronic endoscope system 1z includes the electronic endoscope 100, a processor 200z, and the monitor 300. The electronic endoscope system 1z of the first variation has the same configuration as the electronic endoscope system 1 of the above embodiment, with the exception that the processor 200z does not have the shutter control circuit 220 or the shutter portion 240.

The following describes operations of the electronic endoscope system 1z in various observation modes according to the first variation.

Normal Observation Mode

The following describes operations of the electronic endoscope system 1z in the normal observation mode according to the first variation.

In the normal observation mode, the white LED 206A is on at all times, and the white LED 206B and the purple LED 210B are off at all times. For this reason, the subject is irradiated by white light.

The solid-state image sensor 108 images the subject irradiated by white light, and outputs the image signal to the pre-stage signal processing circuit 226 via the driver signal processing circuit 110. The image signal is processed by the pre-stage signal processing circuit 226, the image memory 228, and the post-stage signal processing circuit 230 and then output to the monitor 300, and thus a normal color image of the subject is displayed on the display screen of the monitor 300.

Note that the purple LED 210B may be on at all times in the normal observation mode in order to improve color rendering.

Special Observation Mode

The following describes operations of the electronic endoscope system 1z in the special observation mode according to the first variation.

In the special observation mode, the white LED 206A is off at all times, and the white LED 206B and the purple LED 210B are on at all times. For this reason, the subject is irradiated by special light filtered by the narrow-band light filter 216B.

The solid-state image sensor 108 images the subject irradiated by special light, and outputs the image signal to the pre-stage signal processing circuit 226 via the driver signal processing circuit 110. For this reason, the image signal is processed by the pre-stage signal processing circuit 226, the image memory 228, and the post-stage signal processing circuit 230 and then output to the monitor 300, and thus a spectral image in which a specific biological structure is enhanced is displayed on the display screen of the monitor 300.

Twin Observation Mode

The following describes operations of the electronic endoscope system 1z in the twin observation mode according to the first variation.

The system controller 202 alternatingly turns on the light sources of the first and second light source portions, thus operating as a control portion for alternatingly causing the two types of light to enter the first and second light guiding members. In the twin observation mode, the white LED 206A is alternatingly turned on and off by the system controller 202 in accordance with a timing synchronized with the frame cycle (one frame at a time). The white LED 206B and the purple LED 210B are also alternatingly turned on and off by the system controller 202 in accordance with a timing synchronized with the frame cycle (one frame at a time). More specifically, in a frame in which the white LED 206A is turned on, the white LED 206B and the purple LED 210B are turned off, and in a frame in which the white LED 206A is turned off, the white LED 206B and the purple LED 210B are turned on.

For this reason, the subject is alternatingly irradiated by white light and special light at a timing synchronized with the frame cycle (one frame at a time).

In one frame, the solid-state image sensor 108 images the subject irradiated by white light and outputs the image signal to the pre-stage signal processing circuit 226 via the driver signal processing circuit 110, and then in the next frame, images the subject irradiated by special light and outputs the image signal to the pre-stage signal processing circuit 226 via the driver signal processing circuit 110. The former and latter image signals are processed by the pre-stage signal processing circuit 226, the image memory 228, and the post-stage signal processing circuit 230 and then output to the monitor 300. Accordingly, a normal color image and a spectral image of the subject are displayed side-by-side on the display screen of the monitor 300.

Note that the purple LED 210B may be on at all times in the twin observation mode in order to improve color rendering in the normal color image.

In this way in the first variation, the shutter control circuit 220 and the shutter portion 240 are not necessary, thus achieving an advantage in terms of manufacturing cost.

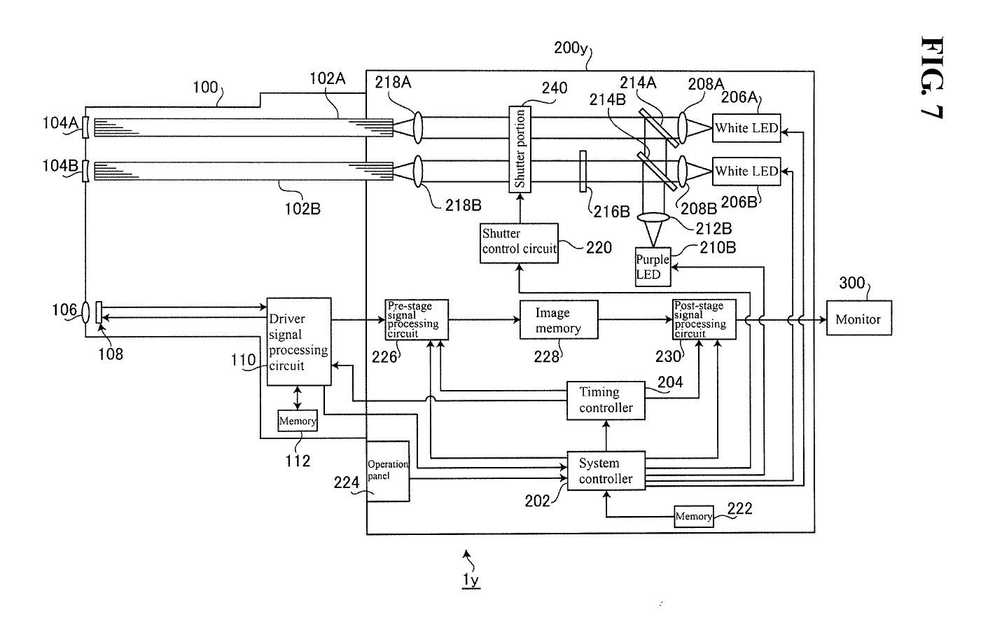

FIG. 7 is a block diagram showing the configuration of an electronic endoscope system 1y according to a second variation of the present embodiment. As shown in FIG. 7, the electronic endoscope system 1y includes the electronic endoscope 100, a processor 200y, and the monitor 300. The electronic endoscope system 1y of the second variation has the same configuration as the electronic endoscope system 1 shown in FIG. 1, with the exception that the processor 200y has a dichroic mirror 214A.

The following describes operations of the electronic endoscope system 1y in various observation modes according to the second variation.

Normal Observation Mode

The following describes operations of the electronic endoscope system 1y in the normal observation mode according to the second variation.

In the normal observation mode, the white LEDs 206A and 206B and the purple LED 210B are on at all times. Also, the rotating disk 241 is stopped in the white light transmission state.

The dichroic mirror 214B simultaneously transmits 50% and reflects 50% of the purple light emitted by the purple LED 210B. For this reason, the portion of the purple light that passed through the dichroic mirror 214B is incident on the dichroic mirror 214A. The purple light that is incident on the dichroic mirror 214A and the white light emitted by the white LED 206A are combined by the dichroic mirror 214A (become superimposed light that has the spectral characteristics shown in FIG. 2(c)), then pass through the rotating disk 241 (opening 241a) and irradiate the subject via the condensing lens 218A, the LCB 102A, and the light distribution lens 104A.

In other words, the subject is irradiated by superimposed light. An image signal of the subject irradiated by superimposed light is processed such that a normal color image of the subject having improved color rendering is displayed on the display screen of the monitor 300. Note that the purple light reflected by the dichroic mirror 214B and the white light emitted by the white LED 206B are filtered by the narrow-band light filter 216B, but are blocked by the rotating disk 241 and do not irradiate the subject.

Special Observation Mode

The following describes operations of the electronic endoscope system 1y in the special observation mode according to the second variation.

In the special observation mode, the white LEDs 206A and 206B and the purple LED 210B are on at all times. Also, the rotating disk 241 is stopped in the special light transmission state. For this reason, white light and purple light emitted by the white LED 206B and the purple LED 210B is filtered by the narrow-band light filter 216B, passes through the rotating disk 241 (opening 241a), and irradiates the subject via the condensing lens 218B, the LCB 102B, and the light distribution lens 104B.

In other words, the subject is irradiated by special light. Note that the purple light reflected by the dichroic mirror 214A and the white light emitted by the white LED 206A are blocked by the rotating disk 241 and do not irradiate the subject. An image signal of the subject irradiated by special light is processed such that a spectral image in which a specific biological structure is enhanced is displayed on the display screen of the monitor 300.

Twin Observation Mode

The following describes operations of the electronic endoscope system 1y in the twin observation mode according to the second variation.

In the twin observation mode, the white LED 206A is alternatingly turned on and off in accordance with a timing synchronized with the frame cycle (one frame at a time). The white LED 206B is also alternatingly turned on and off in accordance with a timing synchronized with the frame cycle (one frame at a time). More specifically, in a frame in which the white LED 206A is turned on, the white LED 206B is turned off, and in a frame in which the white LED 206A is turned off, the white LED 206B is turned on. Also, the purple LED 210B is on at all times. Also, the rotating disk 241 rotates about the rotation shaft 244 such that the position of the opening 241a alternatingly switches between the light path of superimposed light and the light path of special light at a timing synchronized with the frame cycle (one frame at a time). More specifically, the opening 241a is arranged in the light path of superimposed light in a frame in which the white LED 206A is turned on, and is arranged in the light path of special light in a frame in which the white LED 206B is turned on. For this reason, the subject is alternatingly irradiated by superimposed light and special light at a timing synchronized with the frame cycle (one frame at a time). Image signals of the subject irradiated by these types of light are processed such that an image including a normal color image and a spectral image side-by-side is displayed on the display screen of the monitor 300. The subject is irradiated by superimposed light in the normal color image, and therefore color rendering in the normal color image is improved in the twin observation mode as well.

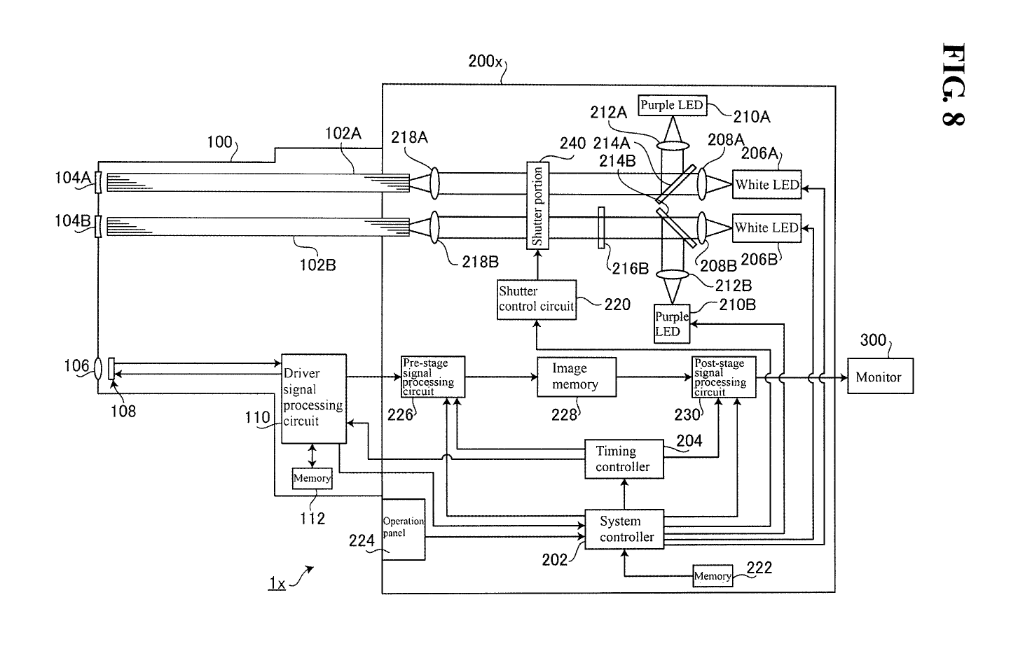

FIG. 8 is a block diagram showing the configuration of an electronic endoscope system 1x according to a third variation of the present embodiment. As shown in FIG. 8, the electronic endoscope system 1x includes the electronic endoscope 100, a processor 200x, and the monitor 300. The electronic endoscope system 1x according to the third variation has the same configuration as the electronic endoscope system 1 shown in FIG. 1, with the exception that the processor 200x has a purple LED 210A, a collimator lens 212A, and a dichroic mirror 214A.

The following describes operations of the electronic endoscope system 1x in various observation modes according to the third variation.

Normal Observation Mode

The following describes operations of the electronic endoscope system 1x in the normal observation mode according to the third variation.

In the normal observation mode, the white LEDs 206A and 206B and the purple LEDs 210A and 210B are on at all times. Also, the rotating disk 241 is stopped in the white light transmission state. The purple light emitted by the purple LED 210A passes through the collimator lens 212A, is reflected by the dichroic mirror 214A, and is combined with white light emitted by the white LED 206A (obtaining superimposed light that has the spectral characteristics shown in FIG. 2(c)), then passes through the rotating disk 241 (opening 241a) and irradiates the subject via the condensing lens 218A, the LCB 102A, and the light distribution lens 104A.

In other words, the subject is irradiated by superimposed light. An image signal of the subject irradiated by superimposed light is processed such that a normal color image of the subject having improved color rendering is displayed on the display screen of the monitor 300. Note that white light and purple light emitted by the white LED 206B and the purple LED 210B are filtered by the narrow-band light filter 216B, but are blocked by the rotating disk 241 and therefore do not irradiate the subject.

Special Observation Mode

The following describes operations of the electronic endoscope system 1x in the special observation mode according to the third variation.

In the special observation mode, the white LEDs 206A and 206B and the purple LEDs 210A and 210B are on at all times. Also, the rotating disk 241 is stopped in the special light transmission state. For this reason, white light and purple light emitted by the white LED 206B and the purple LED 210B is filtered by the narrowband light filter 216B passes through the rotating disk 241 (opening 241a), and irradiates the subject via the condensing lens 218B, the LCB 102B, and the light distribution lens 104B.

In other words, the subject is irradiated by special light. Note that white light and purple light emitted by the white LED 206A and the purple LED 210A are blocked by the rotating disk 241 and do not irradiate the subject. An image signal of the subject irradiated by special light is processed such that a spectral image in which a specific biological structure is enhanced is displayed on the display screen of the monitor 300.

Twin Observation Mode

The following describes operations of the electronic endoscope system 1x in the twin observation mode according to the third variation.

In the twin observation mode, the white LED 206A and the purple LED 210A are alternatingly turned on and off in accordance with a timing synchronized with the frame cycle (one frame at a time). The white LED 206B and the purple LED 210B are also alternatingly turned on and off in accordance with a dining synchronized with the frame cycle (one frame at a time). More specifically, in a frame in which the white LED 206A and the purple LED 210A are turned on, the white LED 206B and the purple LED 210B are turned off, and in a frame in which the white LED 206A and the purple LED 210A are turned off, the white LED 206B and the purple LED 210B are turned on. Also, the rotating disk 241 rotates about the rotation shaft 244 such that the position of the opening 241a alternatingly switches between the light path of superimposed light and the light path of special light at a timing synchronized with the frame cycle (one frame at a time). More specifically, the opening 241a is arranged in the light path of superimposed light in a frame in which the white LED 206A is turned on, and is arranged in the light path of special light in a frame in which the white LED 206B is turned on. For this reason, the subject is alternatingly irradiated by superimposed light and special light at a timing synchronized with the frame cycle (one frame at a time). Image signals of the subject irradiated by these types of light are processed such that an image including a normal color image and a spectral image side-by-side is displayed on the display screen of the monitor 300. The subject is irradiated by superimposed light in the normal color image, and therefore color rendering in the normal color image is improved in the twin observation mode as well.

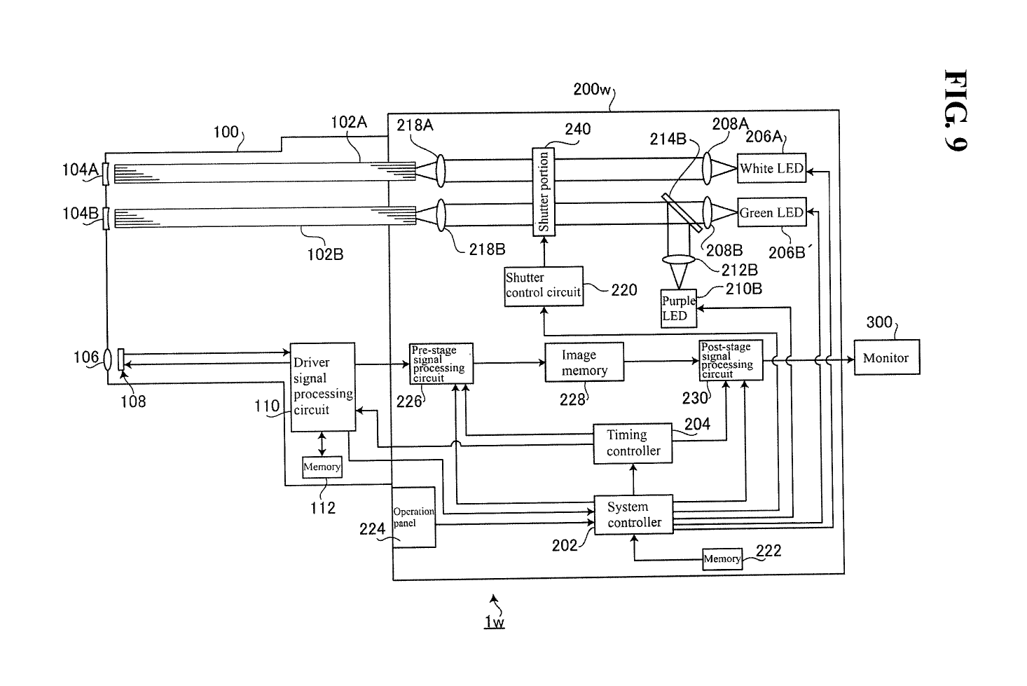

FIG. 9 is a block diagram showing the configuration of an electronic endoscope system 1w according to a fourth variation of the present embodiment. As shown in FIG. 9, the electronic endoscope system 1w includes the electronic endoscope 100, a processor 200w, and the monitor 300. The electronic endoscope system 1w according to the fourth variation has the same configuration as the electronic endoscope system 1 shown in FIG. 1, with the exception that the processor 200w has a green LED 206B′ instead of the white LED 206B, and does not have the narrow-band light filter 216B.

The following describes operations of the electronic endoscope system 1w in various observation modes according to the fourth variation.

Normal Observation Mode

The following describes operations of the electronic endoscope system 1w in the normal observation mode according to the fourth variation.

In the normal observation mode, the white LED 206A, the green LED 206B′, and the purple LED 210B are on at all times. Also, the rotating disk 241 is stopped in the white light transmission state. For this reason, white light emitted by the white LED 206A passes through the rotating disk 241 (opening 241a), and irradiates the subject via the condensing lens 218A, the LCB 102A, and the light distribution lens 104A. On the other hand, green light and purple light emitted by the green LED 206B′ and the purple LED 210B are blocked by the rotating disk 241 and do not irradiate the subject. An image signal of the subject irradiated by white light is processed such that a normal color image of the subject is displayed on the display screen of the monitor 300.

Special Observation Mode

The following describes operations of the electronic endoscope system 1w in the special observation mode according to the fourth variation.

In the special observation mode, the white LED 206A, the green LED 206B′, and the purple LED 210B are on at all times. Also, the rotating disk 241 is stopped in the special light transmission state. For this reason, green light and purple light emitted by the green LED 206B′ and the purple LED 210B pass through the rotating disk 241 (opening 241a) and irradiate the subject via the condensing lens 218B, the LCB 102B, and the light distribution lens 104B.

In other words, the subject is irradiated by light that is a combination of green light and purple light and has characteristics approximating the spectral characteristics shown in FIG. 3(b). Note that white light emitted by the white LED 206A is blocked by the rotating disk 241, and therefore does not irradiate the subject. An image signal of the subject irradiated by light having the above-described characteristics is processed such that a spectral image in which a specific biological structure is enhanced is displayed on the display screen of the monitor 300.

Twin Observation Mode

The following describes operations of the electronic endoscope system 1w in the twin observation mode according to the fourth variation.

In the twin observation mode, the white LED 206A is alternatingly turned on and off in accordance with a timing synchronized with the frame cycle (one frame at a time). The green LED 206B′ and the purple LED 210B are also alternatingly turned on and off in accordance with a timing synchronized with the frame cycle (one frame at a time). More specifically, in a frame in which the white LED 206A is turned on, the green LED 206B′ and the purple LED 210B are turned off, and in a frame in which the white LED 206A is turned off, the green LED 206B′ and the purple LED 210B are turned on. Also, the rotating disk 241 rotates about the rotation shaft 244 such that the position of the opening 241a alternatingly switches between the light path of white light and the light path of special light (green light+purple light) at a timing synchronized with the frame cycle (one frame at a time). More specifically, the opening 241a is arranged in the light path of white light in a frame in which the white LED 206A is turned on, and is arranged in the light path of special light (green light+purple light) in a frame in which the green LED 206B′ is turned on. For this reason, the subject is alternatingly irradiated by white light and light having characteristics approximating the spectral characteristics shown in FIG. 3(b) at a timing synchronized with the frame cycle (one frame at a time). Image signals of the subject irradiated by these types of light are processed such that an image including a normal color image and a spectral image side-by-side is displayed on the display screen of the monitor 300. The electronic endoscope system 1w according to the fourth variation has no need for the narrow-band light filter 216B, and thus has a configuration that is advantageous to cost reduction.

Note that a configuration further including a red LED is conceivable as a further variation of the fourth variation. In this case, the red LED, the green LED 206B′, and the purple LED 210B can be used to irradiate the subject with light that has characteristics approximating the spectral characteristics shown in FIG. 3(a). Accordingly, a spectral image different from those of the above embodiment, variations, and the like is obtained.

Also, although the example of a configuration having the shutter control circuit 220 and the shutter portion 240 is given in the third and fourth variations, in another variation, this configuration may be replaced with a configuration not including the shutter control circuit 220 or the shutter portion 240 similarly to the first variation.

1. An endoscope system comprising:
a first light source that emits first light;
a first light guide that guides the first light received from the first light source toward a subject;
a second light source that emits second light having a different wavelength region from the first light;
a second light guide that guides the second light received from the second light source toward the subject; and
a blocker that alternatingly blocks the first light traveling from the first light source toward the first light guide and the second light traveling from the second light source toward the second light guide.

2. The endoscope system according to claim 1, wherein the blocker alternatingly blocks the first light and the second light in accordance with a timing synchronized with a predetermined imaging cycle.

3. An endoscope system comprising:
a first light source that emits first light;
a first light guide that guides the first light received from the first light source toward a subject;
a second light source that emits second light having a different wavelength region from the first light;
a second light guide that guides the second light received from the second light source toward the subject; and
a controller that, by alternatingly turning on a light source of the first light source portion and a light source of the second light source, alternatingly allows the first light and the second light to enter the first light guide and the second light guide.

4. The endoscope system according to claim 3, wherein the controller alternatingly turns on and off the light source of the first light source and the light source of the second light source in accordance with a timing synchronized with a predetermined imaging cycle.

5. The endoscope system according to claim 1,
wherein the first light source has a light source that emits the first light, and
the second light source has
a light source that emits third light, and
an optical filter that filters the third light to obtain the second light.
